(19) United States
(12) Patent Application Publication
(10) Pub. No.: US 2019/0380984 A1
(43) Pub. Date: Dec. 19, 2019

(54) COMPOSITION CONTAINING MELDONIUM DIHYDRATE FOR THE PREVENTION AND TREATMENT OF NEURODEGNERATIVE DISEASES

(71) Applicant: David Louis Bersohn, Elk Grove, CA (US)

(72) Inventor: David Louis Bersohn, Elk Grove, CA (US)

(21) Appl. No.: 16/010,226

(22) Filed: Jun. 15, 2018

(51) Int. Cl. A61K 31/195; A61P 25/28; A61K 9/00; A61K 36/42

(52) U.S. Cl. CPC A61K 31/195; A61P 25/28; A61K 36/42; A61K 9/0053; A61K 9/0019

(57) ABSTRACT

The present invention relates to the novel use of Medonium dihydrate (MDI) with natural antioxidants, such as lycopene, beta-carotene and lutein, for the prevention and treatment of cebrovascular ischemia and neurodegenerative diseases, such as Alzheimer's disease.

### CROSS-REFERENCE TO RELATED APPLICATIONS

This application is related in part with U.S. Pat. No. 6,770,582 B2 and application 15916252 filed 8 Mar. 2018.

### STATEMENT REGARDING FEDERALLY SPONSORED RESEARCH OR DEVELOPMENT

Not Applicable

### BACKGROUND OF THE INVENTION

### 1. Field of Invention

The present invention relates to pharmaceuticals, nutra-ceuticals, or dietary supplements particularly to a method and composition to prevent and treat diseases.

### 2. Prior Art

It is generally accepted that chronic cerebrovascular ischemia is an important disorder leading to progression of functional neurological deficiency and cognitive impairment. The main cause of this condition is stenosis of the cerebral arteries. Alzheimer's disease (AD) is the commonest neurodegenerative disorder resulting ultimately in dementia, a stage marked by a loss of previously acquired intellectual skill and independent occupational and social function. AD is the most common age-related dementia, afflicting nearly 6 million people in the U.S., increasing to 15 million in 2050. The need for a new approach to treat Alzheimer's is urgent.

Meldonium dihydrate (MDI) with chemical nomenclature of 3-(2,2,2-Trimethylhydrazinium)propionate dihydrate) also known as Mildronate, has been used as a cardioprotective drug for the treatment of cardiovascular diseases for over 30 years in Latvia and 15 other countries. This compound was first synthesized in 1970 by the Latvian Chemist Ivars Kalvins and colleagues at the Latvian Institute of Organic Synthesis, and now is manufactured by the Latvian pharmaceutical company Grindeks and several generic manufacturers.

The compound and its cardioprotective properties are described previously (U.S. Pat. Nos. 4,481,218, 4,451,485 A, 9,573,882, 9,278,907, 8,940,793 and 7,223,797). Due to these properties, MDI is extensively applied in medicine as an anti-ischemic, stress-protective and cardioprotective drug in treating various cardio-vascular diseases and other pathologies involving tissue ischemia (Karpov R. S. et al., 2000). In the treatment of cardiovascular diseases the mechanism of action of MDI is based on limitation of carnitine biosynthesis rate and related to long-chain fatty acid transport limitation through mitochondria membranes (Dambrova et al., 2002).

The main mechanism of action of MDI as a cardiovascular drug is based on the regulation of energy metabolism pathways of free fatty acid transport inhibition through the cell's main powerhouse: mitochondria, which happens indirectly when MDI inhibits enzymes gamma-butyrobetaine hydroxylase and carnitine/organic cation transporter type 2 (OCTN2). Both enzymes are involved in the synthesis of L-carnitine, and by inhibiting the enzymes the L-carnitine levels are lowered. The decline of L-carnitine stimulates glucose metabolism and decreases concentrations of L-carnitine related toxic metabolites, such as the trimethyl-N-oxide and long chain acylcarnitines. By blocking the highly oxygen-consuming process of fatty-acid oxidation, MDI has an acute effect on cardiovascular ischemia. Long term use has been shown to reduce blood glucose concentrations, exhibit cardioprotective effects and prevent or reduce the severity of diabetic complications.

Although MDI has been marketed as a cardioprotective drug, it is a small compound with charged nitrogen and oxygen atoms and can cross the blood brain barrier. It was reported that MDI has learning- and memory-enhancing properties that were shown to correlate with the proliferation of neural progenitor cells and activation of transcription factors in trained rats (Klusa et al., 2013). Ischemia results in tissue damage in a process known as ischemic cascade. The damage is the result of the build-up of metabolic waste products, inability to maintain cell membranes, mitochondrial damage, and eventual leakage of autolyzing proteolytic enzymes into the cell and surrounding tissues. MDI has been shown to block this highly oxygen-consuming process. Alternatively the compound acts via stimulation of nitric oxide (NO) production in the vascular endothelium (Sjakste et al., 2005). Cerebral hypoperfusion due to impaired bioavailability of nitric oxide (NO) synthesized by endothelial nitric oxide synthase and neuronal nitric oxide synthase leads to cognitive decline and neurodegeneration in Alzheimer's disease. Although the brain does not use fatty acid as a source of energy, the neuroprotective functions of MDI acutely and in long-term might be explained by the following actions: blocks the highly oxygen-consuming process of fatty acid metabolism and in turn spares oxygen to highly metabolically active tissues (heart, brain); prevents accumulation of cytotoxic intermediate products of fatty acid β-oxidation. increases ketone body, the main energy source for the brain, due to the use of acetyl-CoA for ketone body synthesis (instead of for fatty-acid metabolism); and stimulates nitric oxide production in the vascular endothelium thus prevents cerebral hypoperfusion and protecting nerve cells or fibers against pathogenic factors associated with cerebral ischemia, trauma, and hemorrhage (Sjakste et al., 2005).

MDI is available on-line and has been used by athletes for performance enhancing (https://nootropicsreviewed.com/meldonium-benefits-side-effects/). In the US, MDI has not been approved for any therapeutic use, and is not a controlled substance.

Oxidative stress has been implicated in the pathogenesis of Alzheimer's disease (AD). Several studies indicate association between cognitive decline and lower plasma level of antioxidants (Mullan et al., Cho et al.)

Momordica cochinchinensis Spreng. (Gac, redmelon) is a fruit originated in Asia, that contains high concentrations of antioxidant carotenoids (U.S. Pat. No. 6,770,580 B2, Vuong, 2004). It is a plausible strategy in combating AD by using antioxidants and this strategy had been put forth in several inventions (U.S. Pat. Nos. 9,989,541; 9,968,576; 9,950,019). None of those inventions described the use of Gac or carotenoids as a source of antioxidants for neurological disorders. The main classes of exogenous antioxidants include polyphenols, vitamins, and carotenoids. The carotenoid class can be further subdivided into carotenes and xanthophylls. The subclass of carotenes consists of α-carotene, β-carotene, and lycopene, whereas the xanthophylls include lutein, zeaxanthin, and β-cryptoxanthin. Lycopene is the most predominant carotenoid, accounting for more than half of total carotenoid in human serum. Because of its unsaturated chemical structure, lycopene has the most powerful antioxidant properties among all serum carotenoids with an oxygen quenching ability twice that of 0-carotene and 10 times higher than α-tocopherol. In a case-control study, lycopene demonstrated the largest significant effect in AD patients The study showed lower levels of all plasma lipophylic antioxidants among AD patients (compared to controls) (Mullan et al.). Lycopene, has been demonstrated to reduce neuroinflammatory phenotypes, depression-like behaviors, and inflammation-induced cognitive function defects in murine models. Neuroinflammation is a local response of the nervous system during neurodegeneration, trauma, and autoimmune disorders. A variety of cell types, including astrocytes, microglia, vascular cells, neutrophils, and macrophages, are involved in neuroinflammation. Growing evidence suggests that neuroinflammation is one of the pathological features of many neurodegenerative disorders. It has been shown that lutein reduces lipid peroxidation and proinflammatory cytokine release by suppressing the activation of the nuclear factor-κB (NF-κB) pathway in the presence of a variety of oxidative stressors. It has also been demonstrated that carotenoids are able to suppress the production of proinflammatory cytokines and nitric oxide by lipopolysaccharide, interferon γ, and β-amyloid (Aβ) stimulation in microglial cells. As a whole, cellular and animal models have revealed that carotenoids are potent anti-inflammatory agents in the nervous system and act through the suppression of inflammation pathways. Gac contains high concentrations of carotenoids, particularly carotenoid antioxidants (lycopene, beta-carotene and lutein). A method using no chemicals to extract beta-carotene from momordica cochinchinensis (Gac fruit) was described by Vuong in U.S. Pat. No. 6,770,585 filed Aug. 2, 2002. This patent claimed a process to produce an oil rich in carotenoids to be used as a safe source of dietary supplement of vitamin A. A process filed subsequently by Ishida et al. (U.S. Pat. No. 7,572,468) described a process using chemincal solvent to extract carotenoids from plants, including Gac fruit. In US Patent 20070212433) Schmid et al. described beverage compositions using Gac fruit in combination with Cili fruit extract, Siberian pineapple extract, and a Wolfberry extract. In U.S. Pat. No. 5,942,233 Chang, Shan described the use of momordica cochinchinensis as one of the component in a paste that is useful for re-etablishing of vital energy invigorating of blood circulation. U.S. Pat. No. 8,017,147, described compositions for the prevention of cardiovascular disease, alzheimer's disease, diabetes, and regulation and reduction of blood sugar and insulin resistance. The inventor mistakenly listed “Momordica chinensis” as a synonym of Bitter Melon (Momordica charantia). U.S. Pat. No. 8,668,942 (Vuong, 2014) described the use of the oil extract from momordica cochinchinensis in skin anti-oxidant enhancing formulations. None of the above patents described the use of the antioxidants in this fruit for the prevention of neurodegenerative diseases.

### CITED REFERENCES

### U.S. Patent

| | | |
|---|---|---|
| 9,573,882 | Feb. 21, 2017 | Kalvins, et al. |
| 9,278,907 | Mar. 8, 2016 | Kalvins, et al. |
| 8,940,793 | Jan. 27, 2015 | Kalvins, et al. |
| 7,223,797 | May 29, 2007 | Kalvinsh, et al. |
| 9,989,541 | Jun. 5, 2018 | Shi, et al. |
| 9,968,576 | May 15, 2018 | Yim, et al. |
| 9,950,019 | Apr. 24, 2018 | Li, et al. |
| 9,974,801 | May 22, 2018 | McGavern,et al. |
| 9,962,426 | May 8, 2018 | Weeber, et al. |
| 6,770,585 | Aug. 3, 2004 | Vuong |
| 8,868,942 | Mar. 11, 2014 | Vuong |
| 20070212433 | June 2004 | Schmid et al. |
| 7,572,468 | Aug. 11, 2009 | Ishida, et al. |
| 5,942,233 | | Chang |

### International Patents

U.S. Pat. No. 8,017,147
References (cited)
Merck Manual Alzheimers disease, accessed Oct. 30, 2016.
Merck Manual Brain infections accessed Oct. 30, 2016.
Uttara et al. Curr Neuropharmacol 2009; 7(1):65-74.
Cole et al. (Neurobiol Aging May-June 2000; 21(3):383-421.
Karpov R. S. et al., Clinical Efficacy and Safety of Mildronate in Patients With Ischemic Heart Disease and Chronic Heart Failure. Kardiologiya. 2000, no. 6, p. 69-74.
Vetra A, Shefere M, Skarda I, Matveja L, Kalvinsh I. Significance of mildronate for improvement of results of early rehabilitation results of neurological patients. Latvijas Arstu Zurnals 1999; 12:33-37 (in Latvian).
Beitnere, Thomas van Groen et. al. Mildronate Improves Cognition and Reduces Amyloid-b Pathology in Transgenic Alzheimer's Disease Mice, Journal of Neuroscience Research 92:338-346 (2014)
Gaidar B V, Parfenov V E, Vainshtein G B. Ways to optimize the cerebral circulation during extreme actions on the brain. Fiziol Zh Sechenova 1989; 75:1568-1575 (in Russian)).
Beketov A I, Mametova A N, Polevik I V, Sapegin I D. Comparative characteristics of cerebrovascular protective effects of mildronate, riboxine, and their combination during modeling of cerebral hemodynamics disturbance. Éksp Klin Farmakol 2000; 63:18-21 (in Russian)).
Germane S. Experimental study of mildronate effect on the central nervous system. Éksp Klin Farmakoter (Riga) 1991; Issue 19:44-50 (in Russian)).
Germane S., Berzina D. Effect of mildronate on catecholamine level and somatic manifestations in white rats organs under stress. Éksp Klin Farmakoter (Riga) 1991; Issue 19:51-56 (in Russian)).
Vinichuk S M. The efficacy of the mildronate treatment of patients with ischemic stroke. Vrach Delo 1991; 7:77-79 (in Russian)).
Enina G, Timofeeva T, Egere D, Majore I. Medicinal effects and indications to mildronate application in neuroangiologic practice. Éksp Klin Farmakoter (Riga) 1991; Issue 19:164-171 (in Russian)).
Dziak L A, Golik V A. Use of mildronate for the treatment of patients with circulatory encephalopathy against a background of stenosis of major arteries of the head. Lik Sprava 2003; 5-6:98-101 (in Russian)). Abeuov B A, Raimkulov B N, Mitrokhin D A, et al. Condition of the higher brain functions in patients with dyscirculatory encephalopathy treated with mildronate. Meditsina 2004; 2:78-81 (in Russian).
Suslina Z A, Fedorova T N, Maksimova Mlu, Kim E K. Antioxidant activity of mildronate and L-carnitine in the treatment of patients with cerebrovascular diseases. Éksp Klin Farmakol 2003; 66:32-35 (in Russian). Petersone I, Veveris M, Berzina D, Kalnciema V, Lepika V, Eglite I. Acute and chronic toxicity of mildronate. Éksp Klin Farmakoter (Riga) 1991; Issue 19:67-71 (in Russian)).
Hayashi Y, Muranaka Y, Kirimoto T, Asaka N, Miyake H, Matsuura N. Effects of MET-88, a gamma-butyrobetaine hydroxylase inhibitor, on tissue carnitine and lipid levels in rats. Biol Pharm Bull 2000; 236: 770-773),
Kalvinysh Ila, Anisimov V N, Khovanova E M, Ugvinenko E G, Tolcheev IuD. Absence of mutagenic and carcinogenic properties in mildronate. Vopr Onkol 1999; 45:279-282 (in Russian)).
VUONG, L., Chitchumroonchokchai C.; Chapman M.; Ishida B.; King J.; Failla M. (2003), High bioaccessibility of carotenes and lycopenes in gac oil and gac fruit aril. The FASEB Journal. Mullan et al. Alzhmeimer's and Dementia, 3, 2017, 432-439.
Cho et al., Recent Advances in Studies on the Therapeutic Potential of Dietary Carotenoids in Neurodegenerative Diseases; Oxidative Medicine and Cellular Longevity Volume 2018, Article ID 4120458, 13 pages

### 7. Definition of Terms

In describing and claiming the present invention, the following terminology will be used.

The singular forms “a,” “an,” and “the” include plural referents unless the context clearly dictates otherwise. Thus, for example, reference to “a carrier” includes reference to one or more of such carriers, and reference to “an excipient” includes reference to one or more of such excipients.

As used herein, “formulation” and “composition” may be used interchangeably herein, and refer to a combination of two or more elements, or substances. In some embodiments a composition may include an active agent and a carrier.

As used herein, “effective amount” refers to an amount of an ingredient, namely composition, is sufficient to be able to detect by available analytical tool by qualified persons.

As used herein, the term “Meldonate dihydrate”, “Mildronate”, “MDI” may be used interchangeably herein.

The term Redmelon or Gacmelon or Gac or “Gac fruit” or “Gac plant” may be used interchangeably herein.

The term “Gac Fruit,” “Gac Extract,” “Gac fruit extract,” “Gac Fruit Puree,” “Gac powder,” “Gac oil,” “Gac fruit oil” refer to a product made from the fruit of all strains and hybrids of the plant Momordica cochinchinensis Spreng, or of plants significantly related thereto, grown anywhere in the world including blends, mixtures, and combinations of such strains and relatives.

### OBJECTS AND ADVANTAGES

The objects of this invention relate to methods and compositions to prevent and slow the progress of neuro-degenerative diseases. The compositions described herein contain Meldonium dihydrate, a known compound that has been used widely as a cardiovascular drug, and by athletes to improve performance. The compositions also contain antioxidant carotenoids, which have been shown to protect cells from oxidative damages. The methods and compositions described herein are safe for consumers and can slow the disease progress, improve the conditions and the quality of life of patients of neurodegenerative diseases such as Alzheimer's disease, or chronic cerebrovascular ischemia.

### BRIEF SUMMARY OF THE INVENTION

The present invention relates to the novel use of Medonium dihydrate (MDI) with or without natural or synthetic antioxidants, such as lycopene, beta-carotene and lutein, for the prevention and treatment of cebrovascular ischemia and neurodegenerative diseases, such as Alzheimer's disease. Meldonium dihydrate is a small molecule, a carnitine congener involved in fatty acid oxidation in the mitochondria and oxygen consumption, and has been used widely as a cardiovascular drug and performance enhancing drug by athletes. Research studies indicated that MDI has learning- and memory-enhancing properties that were shown to correlate with the proliferation of neural progenitor cells and activation of transcription factors. MDI is also involved in the regulation of Nitric Oxide production, and hemodynamics of the cerebrovascular system, and in glucose metabolism and ketone body production, which is the source of energy for the brain. In one embodiment, given to patients with brain circulation disorders. MDI improved patients' mood; they became more active, their motor dysfunction decreased, and asthenia, dizziness and nausea became less pronounced. Oxidative stress damage has been shown to be one of the etiology of neurological diseases. Carotenoid antioxidants have been shown to reduce cellular oxidative damage. Natural carotenoid antioxidants in the compositions can be from fruits, such as momordica cochinchinensis Spreng., the highest and most bioavailable source of lycopene and beta-carotene. The formulation can be used daily as dietary supplement for the prevention of neurodegenerative diseases, or to improve cognitive functions and quality of life for patients of neurological or cerebrovascular disorders.

### BRIEF DESCRIPTION OF THE SEVERAL VIEWS OF THE DRAWING

(Not Applicable)

### DETAILED DESCRIPTION OF THE INVENTION

The present invention is directed to the use of Meldonium dihydrate (MDI) to prevent or lessen the damage of neuro-degenerative diseases. MDI is a carnitine congener, that was discovered and synthesized in 1970, and used widely as a cardiovascular drug. The compound is available from several manufacturers.

It has been shown that the compound can pass through the blood brain barrier and exert neuroprotective properties. In an embodiment, when an effective amount of MDI is given to patients of brain circulation disorders, the patients become more active, their motor dysfunction decreases, and asthenia, dizziness and nausea become less pronounced.

The CNS effects of MDI could be mediated by stimulation of the nitric oxide production in the vascular endothelium by modification of the γ-butyrobetaine esters. The latter are potent cholinomimetics and may activate NOS via acetylcholine receptors or specific γ-butyrobetaine esters receptors. In another embodiment, MDI (25 mg/kg, i.v. administered daily during 14 days after induction of local brain lesions and ischemia in rabbits, facilitated the restoration of cerebral blood flow and vascular reactivity. The effect involved an improvement of restorative processes due to a more rapid normalization of reactivity of cerebral blood vessels.

Accordingly, in accordance with an embodiment, the invention provides a method of treating or preventing a disease or disorder of the central nervous system (CNS) in a patient comprising administering an effective amount of MDI alone or in combination with an effective amount of a reactive oxygen species (ROS) scavenger such as Gac fruit puree to the patient. The disease or disorder of the CNS can be any suitable disease or disorder, for example, those selected from the group consisting of brain injury, inflammation, infection, degeneration of brain cells, stroke, brain edema, tumor, Alzheimer's disease, Parkinson's disease, and multiple sclerosis. In a particular embodiment, the disease or disorder of the CNS is brain injury, and more particularly traumatic brain injury resulting from activity associated with boxing, football, soccer, hockey, armed conflict, or brain surgery.

Impaired sociability and dysregulated social behavior have been implicated as hallmarks not only in AD pathology but also in many psychiatric disorders, such as autism and schizophrenia. In an embodiment, MDI at doses of 50 mg/kg was administered intraperitoneally (i.p.) every day for 28 days to transgenic Alzheimer's disease mice. MDI significantly improved social recognition, cognitive performance and lowered Ab plaque load in hippocampus.

In a particular embodiment, the antioxidants are selected from the group consisting of glutathione, ascorbic acid, lipoic acid, uric acid, carotenes, .alpha.-tocopherol, ubiquinols, and combinations thereof, and more particularly, lycopene, beta-carotene, lutein from the fruit momordica cochinchinensis Spreng. (Gac fruit).

Since neuro-behavioral, particularly cognitive related, problems are a major effect of traumatic brain injury, various methods used to assess cognitive function can be used. Such assessments include, among others, the following: Clinical Dementia Rating Scale (CDR), a dementia staging instrument that classifies cognitive impairment along a continuum from normal aging to mild cognitive impairment to all stages of dementia severity; Folstein Mini-Mental State Exam (MMSE), which is commonly used to measure of orientation and gross cognitive functioning used by physicians and healthcare providers to screen for cognitive decline; and Alzheimer's Disease Assessment Scale-Cognitive (ADAS-C), a test commonly used in detection of dementia and mild cognitive impairment.

Additionally, the presence of traumatic brain injury in a patient can be assessed by standard techniques used by a physician of skill in the art. These include, among others, Glasgow Coma Scale, which is a 15-point test that helps assess the severity of a brain injury by checking patient's ability to follow directions, to blink the eyes or to move extremities; brain imaging techniques, including computer assisted tomography (CAT) scans, which allow visualization of fractures and evidence of bleeding in the brain (hemorrhage), large blood clots (hematomas), bruised brain tissue (contusions), and brain tissue swelling. In embodiments, the brain imaging technique used can be magnetic resonance imaging (MRI), including Susceptibility Weighted Images (SWI), a sensitive method for detecting small hemorrhages in the brain, and Diffusion tensor imaging (DTI), which consists of a minimum of six scans with diffusion gradients placed in an orthogonal manner. In some embodiments, traumatic brain injury can be assessed by measuring intracranial pressure, which can occur by swelling of the brain.

In one embodiment, after MDI treatment, cerebral circulation parameters of patients with an early form of cerebral insufficiency and stage II-III encephalophathy, were evaluated by means of echopulsography.

Additional methods for assessing cognitive impairment from traumatic brain injury can include, among others, various neuropsychological test, such as the following: Wechsler Test of Adult Reading (WTAR), which is a measure of word pronunciation and is a reliable predictor of pre-morbid general intellectual function; Wechsler Adult Intelligence Scale-3 (WAIS-3)-Kaufman tetrad short form, which is used to measure general intellectual functioning; Repeatable Battery for the Assessment of Neuropsychological Status (RBANS), a comprehensive but relatively rapid, standardized measure of neurocognitive functioning in multiple domains, including memory, attention, language, and visuospatial/constructional functions; Trailmaking Test Part A (Trails A), a widely-used measure of cognitive processing and visuomotor speed, and with Part B, also previously employed in studies of mild cognitive impairment (MCI); Trailmaking Test Part B (Trails B), a more complex measure of cognitive processing with executive demands related to mental flexibility and working memory; Controlled Oral Word Association Test (COWAT), a well-known measure of phonemically-controlled verbal fluency, sensitive to cognitive slowing and impairments of executive functioning and routinely employed in dementia assessment and MCI studies; Boston Naming Test (BNT), a visual confrontation naming measure utilized to detect anomia or word-finding difficulties, which are common hallmarks of cognitive decline in elderly populations with mild cognitive impairment or early dementia; Automated Neuropsychological Assessment Metrics (ANAM), a computerized test designed to assess several cognitive domains known to be sensitive to change following concussion, including attention and concentration, reaction time, working memory, new learning and memory, and speed of information processing; and SF-36, which measures eight domains of health, including, physical functioning, role limitations due to physical health, bodily pain, general health perceptions, vitality, social functioning, role limitations due to emotional problems, and mental health.

It is possible that the compositions can be produced as solids, such as powders or granules. The solids can be applied directly or dissolved in water or a biocompatible solvent prior to use to form a solution that is substantially neutral or that has been rendered substantially neutral and that can then be given by mouth.

The composition of MDI and antioxidants can be administered in combination with a pharmaceutically acceptable carrier. The pharmaceutically acceptable carrier (or excipient) is preferably one that is chemically inert to the compound of the invention and one that has no detrimental side effects or toxicity under the conditions of use. The choice of carrier will be determined in part by the particular compound chosen, as well as by the particular method used to administer the composition. Accordingly, there is a wide variety of suitable formulations of the pharmaceutical composition of the present invention.

Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease or condition.

The treatment regimens can vary depending on the severity of the CNS disease or disorder. In one embodiment, MDI is given p.o. or i.v. to patients with an early form of cerebral insufficiency and stage II-III encephalopathy. In another embodiment, the MDI treatment regiment is 10% infusion solution, 10 mL, for up to 10 days followed by tablets, 750 mg/day for 20 days in patient with chronic cerebrovascular ischemia.

The therapeutically effective amount of the compound or compounds administered can vary depending upon the desired effects and the factors noted above. In accordance with an embodiment, examples of drug dosages can be between 10 mg/kg and 60 mg/kg of the subject's body weight.

Unit dosage forms can be formulated based upon the suitable ranges recited above and the subject's body weight. The term “unit dosage form” as used herein refers to a physically discrete unit of therapeutic agent appropriate for the subject to be treated.

The following examples further illustrate the invention but, of course, should not be construed as in any way limiting its scope.

### EXAMPLES

MDI, 25 mg/kg, intravenously, administered daily during 14 days after induction of local brain lesions and ischemia (in rabbits) facilitated the restoration of cerebral blood flow and vascular reactivity.

A pre-treatment to improve hemodynamics, optimize oxygen balance and lessen the damage of brain edema:

Per day: 750 mg MDI+20 g of Gac puree dissolved in 100 mL of purified water

A composition of MDI (10 mL of 5% solution) was administered intravenously to patients with ischemic stroke once a day. It was found that MDI improved cerebral hemodynamics in patients with stroke.

MDI was given p.o. to patients with an early form of cerebral insufficiency and stage II-III encephalopathy. After MDI administration patients became more active, headaches and asthenia diminished, while efficiency increased.

MDI was used in patients with chronic cerebrovascular ischemia (10% infusion solution, 10 mL, for up to 10 days followed by tablets, 750 mg/day and antioxidants (20 g, oral), for 20 days. There was a positive effect on neurological symptoms, hemodynamic, electrophysiological, and neuropsychological characteristics of the patients.

MDI (250 mg intravenously., twice a day for 10 days) in combination with antioxidant carotenoids (20 mg) decreased the incidence of headaches, dizziness, vestibular dysfunction, and insomnia. Patients noted improvement in memory, attention, and cognition.

Administered as add-on therapy, MDI (at a daily dose of 500 mg) increased the resistance of blood serum lipoproteins to peroxidation.

1. Method and composition for the prevention and treatment of neuro-degenerative disease in a subject in need thereof, comprising administration of a pharmaceutical containing an effective amount of Meldonium dihydrate to the subject.

2. The method in claim 1 wherein the effective amount of Meldonium dihydrate is given orally or intravenously, or parenterally.

3. The composition of claim 1, further comprising an effective amount of natural antioxidants, particularly from momordica cochinchinensis Spreng (Gac).

4. The composition of claim 1 further comprising pharmaceutically acceptable carriers, excipients or diluents.

5. A method to improve cognitive functions in subject suffering from Alzheimer's disease and/or dementia, comprising administering a daily dietary supplement containing an effective amount of Meldonium dihydrate

6. The method according to claim 6, wherein the composition might contain natural antioxidants or acceptable food additives
